(19) United States
(12) Patent Application Publication
(10) Pub. No.: US 2010/0248209 A1
(43) Pub. Date: Sep. 30, 2010

(54) Three-dimensional integrated circuit for analyte detection

(76) Inventors: Suman Datta, Beaverton, OR (US); Shriram Ramanathan, Cambridge, MA (US); Jack T. Kavalieros, Portland, OR (US); Justin K. Brask, Portland, OR (US); Brandon Barnett, Beaverton, OR (US)

Correspondence Address: Pillsbury Winthrop Shaw Pittman LLP;(INTEL), P.O. Box 10500, McLean, VA, 22102, US

(21) Appl. No.: 11/478,335

(22) Filed: Jun. 30, 2006

(51) Int. Cl. G01R 27/28; H01L 29/78; H01L 29/66; H01L 21/28; G01N 27/00; C12Q 1/70; C12Q 1/02; G01N 33/92; G01N 33/00; G01N 33/53; G01N 33/68

(52) U.S. Cl. 435/5; 324/649; 257/253; 257/9; 438/49; 422/82.01; 435/29; 436/71; 436/94; 436/501; 436/86; 257/E29.255; 257/E21.158; 257/E29.168; 977/936

(57) ABSTRACT

The embodiments of the invention relate to a device having a first substrate comprising a transistor; a second substrate; an insulating layer in between and adjoining the first and second substrates; and an opening within the second substrate, the opening being aligned with the transistor; wherein the transistor is configured to detect an electrical charge change within the opening. Other embodiments relate to a method including providing a substrate comprising a first part, a second part, and an insulating layer in between and adjoining the first and second parts; fabricating a transistor on the first part; and fabricating an opening within the second part, the opening being aligned with the transistor; wherein the transistor is configured to detect an electrical charge change within the opening.

### RELATED APPLICATIONS

None.

### FIELD OF INVENTION

The embodiments of the invention relate to a device and method for detection of biomolecules such as analytes. Specifically, the embodiments encompass using semiconductor device comprising transistors as electrical sensors in the detection of biomolecules. The invention transcends several scientific disciplines such as, biochemistry, physics, microelectronics, immunology, molecular biology, and medical diagnostics.

### BACKGROUND

Rapid and specific detections of biomolecules and biological cells, such as proteins, DNAs, and RNAs, viruses, peptides, antibodies, antigens, red blood cells, white blood cells, and platelets, have become more and more important to biological assays crucial to fields such as genomics, proteomics, diagnoses, and pathological studies. For example, the rapid and accurate detection of specific antigens and viruses is critical for combating pandemic diseases such as AIDS, flu, and other infectious diseases. Also, due to faster and more specific methods of separating and detecting cells and biomolecules, the molecular-level origins of disease are being elucidated at a rapid pace, potentially ushering in a new era of personalized medicine in which a specific course of therapy is developed for each patient. To fully exploit this expanding knowledge of disease phenotype, new methods for detecting multiple biomolecules (e.g., viruses, DNAs and proteins) simultaneously are increasingly desired and required. The multiplex biomolecule detection methods must be rapid, sensitive, highly parallel, and ideally capable of diagnosing cellular phenotype in vivo.

A specific type of biological assay increasingly used for medical diagnostics, as well as in food and environmental analysis, is immunoassay. An immunoassay is a biochemical test that measures the level of a substance in a biological liquid, such as serum or urine, using the reaction of an antibody its antigen. The assay takes advantage of the specific binding of an antibody to its antigen. Monoclonal antibodies are often used as they only usually bind to one site of a particular molecule, and therefore provide a more specific and accurate test, which is less easily confused by the presence of other molecules. The antibodies picked must have a high affinity for the antigen (if there is antigen available, a very high proportion of it must bind to the antibody). In an immunoassay, both the presence of antigen or antibodies can be measured. For instance, when detecting infection the presence of antibody against the pathogen is measured. For measuring hormones such as insulin, the insulin acts as the antigen.

Conventionally, for numerical results, the response of the fluid being measured must be compared to standards of a known concentration. This is usually done though the plotting of a standard curve on a graph, the position of the curve at response of the unknown is then examined, and so the quantity of the unknown found. The detection of the quantity present of antibody or antigen can be achieved by a variety of methods. One of the most common is to label either the antigen or antibody. The label may consist of an enzyme, radioisotopes, or a fluorophore.

An increasing amount of biological assays, such as immunoassays and gene sequencing, are being carried out on microarrays, such as DNA microarrays or protein microarrays. A microarray is a collection of microscopic spots containing probes, such as DNA or protein spots attached to a solid surface, such as glass, plastic or silicon chip forming an array. Multiple probes can be assembled on a single substrate by techniques well know to one skilled in the art. A probe could bind to an analyte or group or analytes by hybridization. Examples of uses of such an array include, but are not limited to, investigations to determine which genes are active in cancer, investigations to determine which gene differences make a patient have a bad reaction to a drug treatment, investigations for infectious disease, investigations to determine presence of genetic mutation in a patient.

Currently, detection of chemical reaction or binding is accomplished with a multi-step process as shown in FIG. 1. The analyte in the sample is labeled with a fluorescent or other tag (e.g. luminescent, radioactive, dye, etc.). A sample is washed over the array and analytes bind to their complementary probes on the surface due to hybridization. When binding occurs to the probe on the substrate, the label is bound to a location on the substrate. An instrument is used to illuminate the tag creating a spot visible to a reader. Often, fluorescent labels are used and read with an instrument employing laser illumination and a CCD camera to digitize the location and brightness of bound labels.

### BRIEF DESCRIPTION OF THE DRAWINGS

FIG. 1 (prior art) schematically illustrates a method of detection of an analyte with a fluorescent-type tag using a conventional microarray.

FIG. 2 shows a biochemical sensor of the embodiments of the invention. FIG. 2a shows a plan of records a SOI FET used as logic transistors for microprocessor applications. FIG. 2b shows a SOI device can act as a sensor by exposing the bio-chemical to the channel region of the transistor. FIG. 2c illustrates a SOIFET sensor manufactured using 3-D wafer stacking technologies.

FIG. 3 shows a cross-sectional view of a stacked wafer device that could be used as a biosensor in the embodiments of the invention.

FIG. 4 shows a think body SOI device and the effect of the back substrate bias voltage on the channel transport of a p type transistor containing device.

FIG. 5 a microarray having a biochemical sensor of the embodiments of the invention.

FIG. 6 shows a schematic of a method of an assay with on-chip direct detection and digitized readout and analysis.

### DETAILED DESCRIPTION

As used in the specification and claims, the singular forms “a”, “an” and “the” include plural references unless the context clearly dictates otherwise. For example, the term “an array” may include a plurality of arrays unless the context clearly dictates otherwise.

An “electrical sensor,” “biochemical sensor” or “sensor” refers to a substance or device that detects or senses an electrical signal created by movement of electrons, including but not limited to electrical resistance, current, voltage and power.

A “field effect transistor” or FET refers to a transistor that relies on an electric field to control the conductivity of a channel in a semiconductor material. An FET has three terminals, which are commonly known as the gate, drain and source. A voltage applied between the gate and source terminals modulates the current between the source and drain terminals. A small change in gate voltage can cause a large variation in the current from the source to the drain, thus enabling the FET to amplify signals. A field effect transistor (FET) is a transistor that relies on an electric field to control the conductivity of a channel in a semiconductor material. An FET has three terminals, which are known as the gate, drain and source. A voltage applied between the gate and source terminals modulates the current between the source and drain terminals. A small change in gate voltage can cause a large variation in the current from the source to the drain, thus enabling the FET to amplify signals. FETs could be used for weak-signal amplification and can amplify analog or digital signals. They could also be used as voltage-controlled resistors and as sensors in chemical and biological detection.

An “array,” “macroarray” or “microarray” is an intentionally created collection of substances, such as molecules, openings, microcoils, detectors and/or sensors, attached to or fabricated on a substrate or solid surface, such as glass, plastic, silicon chip or other material forming an array. The arrays can be used to measure the expression levels of large numbers, e.g., tens, thousands or millions, of reactions or combinations simultaneously. An array may also contain a small number of substances, e.g., a few or a dozen. The substances in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the pads on the array. A macroarray generally contains pad sizes of about 300 microns or larger and can be easily imaged by gel and blot scanners. A microarray would generally contain pad sizes of less than 300 microns.

“Substrate,” “support” and “solid support” refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain aspects, the solid support(s) will take the form of beads, resins, gels, microspheres, or other geometric configurations.

The term “analyte,” “target” or “target molecule” refers to a molecule of interest that is to be detected and/or analyzed, e.g., a nucleotide, an oligonucleotide, a polynucleotide, a peptide, or a protein. The analyte, target or target molecule could be a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to molecular probes such as chemically modified carbon nanotubes, carbon nanotube bundles, nanowires, nanoclusters or nanoparticles. The target molecule may be a fluorescently labeled antigen, antibody, DNA or RNA. A “bioanalyte” refers to an analyte that is a biomolecule.

The term “capture molecule” refers to a molecule that is immobilized on a surface. The capture molecule generally, but not necessarily, binds to a target or target molecule. The capture molecule is typically an antibody, a nucleotide, an oligonucleotide, a polynucleotide, a peptide, or a protein, but could also be a small molecule, biomolecule, or nanomaterial such as but not necessarily limited to a small molecule that is biologically active, nucleic acids and their sequences, peptides and polypeptides, as well as nanostructure materials chemically modified with biomolecules or small molecules capable of binding to a target molecule that is bound to a probe molecule to form a complex of the capture molecule, target molecule and the probe molecule. In the case of a solid-phase immunoassay, the capture molecule in immobilized on the surface of the substrate and is an antibody specific to the target, an antigen, to be detected. The capture molecule may be fluorescently labeled antibody, protein, DNA or RNA. The capture molecule may or may not be capable of binding to just the target molecule or just the probe molecule.

The term “probe” or “probe molecule” refers to a molecule that binds to a target molecule for the analysis of the target. The probe or probe molecule is generally, but not necessarily, has a known molecular structure or sequence. The probe or probe molecule may or may not be attached to the substrate of the array. The probe or probe molecule is typically an antibody, a nucleotide, an oligonucleotide, a polynucleotide, a peptide, or a protein, including, for example, monoclonal antibody, cDNA or pre-synthesized polynucleotide deposited on the array. Probes molecules are biomolecules capable of undergoing binding or molecular recognition events with target molecules. (In some references, the terms “target” and “probe” are defined opposite to the definitions provided here.) In immunoassays, the probe molecule may be a labeled antibody specific to the target, an antigen, to be analyzed. In such case, the capture molecule, the target molecule and the probe molecule form a “sandwich.” The polynucleotide probes require only the sequence information of genes, and thereby can exploit the genome sequences of an organism. In cDNA arrays, there could be cross-hybridization due to sequence homologies among members of a gene family. Polynucleotide arrays can be specifically designed to differentiate between highly homologous members of a gene family as well as spliced forms of the same gene (exon-specific). Polynucleotide arrays of the embodiment of this invention could also be designed to allow detection of mutations and single nucleotide polymorphism. A probe or probe molecule can be a capture molecule.

A “binding partner,” refers to a molecule or aggregate that has binding affinity for one or more analytes, targets or other molecules. In this sense, a binding partner is either a “capture molecule” or a “probe molecule.” Within the scope of the embodiments of the invention, virtually any molecule or aggregate that has a binding affinity for an analyte or target of interest may be a binding partner, including, but are not limited to, polyclonal antibodies, monoclonal antibodies, single-chain antibodies, chimeric antibodies, humanized antibodies, antibody fragments, oligonucleotides, polynucleotides, nucleic acids, aptamers, nucleic acid ligands and any other known ligand that can bind to at least one target molecule. Although, in certain embodiments a binding partner is specific for binding to a single target, in other embodiments the binding partner may bind to multiple targets that possess similar structures or binding domains.

“Binding” refers to an interaction between two or more substances, such as between a target and a capture or probe molecule, that results in a sufficiently stable complex so as to permit detection of the bound molecule complex. In certain embodiments of the invention, binding may also refer to an interaction between a second molecule and a target.

“Associated with” or “association” refers to a direct or indirect interactions between two or more substances, such as between a target and a capture or probe molecule, that results in a sufficiently stable complex. For example, a molecule or complex of molecules is “associated with” the surface of a substrate when the molecule or complex is either bound to the surface of the substrate directly, through another molecule or substance, or to both. In other words, substances are “associated with” each other when any one member of the substances is directly bound to at least another member of the substances. Additionally, a component of an integrated device is also “associated with” the device. For example, a transistor in an integrated circuit is “associated with” the circuit.

The terms “label,” “tag” and “sensor compound” are used interchangeably to refer to a marker or indicator distinguishable by the observer but not necessarily by the system used to identify an analyte or target. A label may also achieve its effect by undergoing a pre-designed detectable process. Labels are often used in biological assays to be conjugated with, or attached to, an otherwise difficult to detect substance. At the same time, Labels usually do not change or affect the underlining assay process. A label or tag used in biological assays include, but not limited to, a radio-active material, a magnetic material, quantum dot, an enzyme, a liposome-based label, a chromophore, a fluorophore, a dye, a nanoparticle, a quantum dot or quantum well, a composite-organic-inorganic nano-cluster, a colloidal metal particle, or a combination thereof.

The terms “die,” “polymer array chip,” “array,” “array chip,” or “bio-chip” are used interchangeably and refer to a collection of a large number of capture molecules arranged on a shared substrate which could be a portion of a silicon wafer, a nylon strip or a glass slide. The term “DNA array” or “DNA array chip” is used when the array chip is used to analyze a nucleotide. The term “protein array” is used when the array chip is used to analyze a protein.

The term “chip” or “microchip” refers to a microelectronic device made of semiconductor material and having one or more integrated circuits or one or more devices. A “chip” or “microchip” is typically a section of a wafer and made by slicing the wafer. A “chip” or “microchip” may comprise many miniature transistors and other electronic components on a single thin rectangle of silicon, sapphire, germanium, silicon nitride, silicon germanium, or of any other semiconductor material. A microchip can contain dozens, hundreds, or millions of electronic components.

“Micro-Electro-Mechanical System (MEMS)” is the integration of mechanical elements, sensors, actuators, and electronics on a common silicon substrate through microfabrication technology. While the electronics are fabricated using integrated circuit (IC) process sequences (e.g., CMOS, Bipolar, or BICMOS processes), the micromechanical components could be fabricated using compatible “micromachining” processes that selectively etch away parts of the silicon wafer or add new structural layers to form the mechanical and electromechanical devices. Microelectronic integrated circuits can be thought of as the “brains” of a system and MEMS augments this decision-making capability with “eyes” and “arms”, to allow microsystems to sense and control the environment. Sensors gather information from the environment through measuring mechanical, thermal, biological, chemical, optical, and magnetic phenomena. The electronics then process the information derived from the sensors and through some decision making capability direct the actuators to respond by moving, positioning, regulating, pumping, and filtering, thereby controlling the environment for some desired outcome or purpose. Because MEMS devices are manufactured using batch fabrication techniques similar to those used for integrated circuits, unprecedented levels of functionality, reliability, and sophistication can be placed on a small silicon chip at a relatively low cost.

“Microprocessor” is a processor on an integrated circuit (IC) chip. The processor may be one or more processor on one or more IC chip. The chip is typically a silicon chip with thousands of electronic components that serves as a central processing unit (CPU) of a computer or a computing device.

A “macromolecule” or “polymer” comprises two or more monomers covalently joined. The monomers may be joined one at a time or in strings of multiple monomers, ordinarily known as “oligomers.” Thus, for example, one monomer and a string of five monomers may be joined to form a macromolecule or polymer of six monomers. Similarly, a string of fifty monomers may be joined with a string of hundred monomers to form a macromolecule or polymer of one hundred and fifty monomers. The term polymer as used herein includes, for example, both linear and cyclic polymers of nucleic acids, polynucleotides, polynucleotides, polysaccharides, oligosaccharides, proteins, polypeptides, peptides, phospholipids and peptide nucleic acids (PNAs). The peptides include those peptides having either α-, β-, or ω-amino acids. In addition, polymers include heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent upon review of this disclosure.

A “nanomaterial” as used herein refers to a structure, a device or a system having a dimension at the atomic, molecular or macromolecular levels, in the length scale of approximately 1-100 nanometer range. Preferably, a nanomaterial has properties and functions because of the size and can be manipulated and controlled on the atomic level.

The term “biomolecule” refers to any organic molecule that is part of a living organism. Biomolecules includes a nucleotide, a polynucleotide, an oligonucleotide, a peptide, a protein, a ligand, a receptor, among others. A “complex of a biomolecule” refers to a structure made up of two or more types of biomolecules. Examples of a complex of biomolecule include a cell or viral particles. A cell can include bacteria, fungi, animal mammalian cell, for example.

The term “nucleotide” includes deoxynucleotides and analogs thereof. These analogs are those molecules having some structural features in common with a naturally occurring nucleotide such that when incorporated into a polynucleotide sequence, they allow hybridization with a complementary polynucleotide in solution. Typically, these analogs are derived from naturally occurring nucleotides by replacing and/or modifying the base, the ribose or the phosphodiester moiety. The changes can be tailor-made to stabilize or destabilize hybrid formation, or to enhance the specificity of hybridization with a complementary polynucleotide sequence as desired, or to enhance stability of the polynucleotide.

The term “polynucleotide” or “polynucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

An “oligonucleotide” is a polynucleotide having 2 to 20 nucleotides. Analogs also include protected and/or modified monomers as are conventionally used in polynucleotide synthesis. As one of skill in the art is well aware, polynucleotide synthesis uses a variety of base-protected nucleoside derivatives in which one or more of the nitrogen atoms of the purine and pyrimidine moiety are protected by groups such as dimethoxytrityl, benzyl, tert-butyl, isobutyl and the like.

For instance, structural groups are optionally added to the ribose or base of a nucleoside for incorporation into a polynucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary polynucleotides (especially RNA) than their unmodified counterparts. Alternatively, deazapurines and deazapyrimidines in which one or more N atoms of the purine or pyrimidine heterocyclic ring are replaced by C atoms can also be used.

The phosphodiester linkage or “sugar-phosphate backbone” of the polynucleotide can also be substituted or modified, for instance with methyl phosphonates, O-methyl phosphates or phosphororthioates. Another example of a polynucleotide comprising such modified linkages for purposes of this disclosure includes “peptide polynucleotides” in which a polyamide backbone is attached to polynucleotide bases, or modified polynucleotide bases. Peptide polynucleotides which comprise a polyamide backbone and the bases found in naturally occurring nucleotides are commercially available.

Nucleotides with modified bases can also be used in the embodiments of the invention. Some examples of base modifications include 2-aminoadenine, 5-methylcytosine, 5-(propyn-1-yl)cytosine, 5-(propyn-1-yl)uracil, 5-bromouracil, 5-bromocytosine, hydroxymethylcytosine, methyluracil, hydroxymethyluracil, and dihydroxypentyluracil which can be incorporated into polynucleotides in order to modify binding affinity for complementary polynucleotides.

Groups can also be linked to various positions on the nucleoside sugar ring or on the purine or pyrimidine rings which may stabilize the duplex by electrostatic interactions with the negatively charged phosphate backbone, or through interactions in the major and minor groves. For example, adenosine and guanosine nucleotides can be substituted at the N² position with an imidazolyl propyl group, increasing duplex stability. Universal base analogues such as 3-nitropyrrole and 5-nitroindole can also be included. A variety of modified polynucleotides suitable for use in the embodiments of the invention are described in the literature.

When the macromolecule of interest is a peptide, the amino acids can be any amino acids, including α, β, or ω-amino acids. When the amino acids are α-amino acids, either the L-optical isomer or the D-optical isomer may be used. Additionally, unnatural amino acids, for example, β-alanine, phenylglycine and homoarginine are also contemplated by the embodiments of the invention. These amino acids are well-known in the art.

A “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often more than 20 amino acid monomers long.

A “protein” is a long polymer of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies.

The term “sequence” refers to the particular ordering of monomers within a macromolecule and it may be referred to herein as the sequence of the macromolecule.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” For example, hybridization refers to the formation of hybrids between a probe polynucleotide (e.g., a polynucleotide of the invention which may include substitutions, deletion, and/or additions) and a specific target polynucleotide (e.g., an analyte polynucleotide) wherein the probe preferentially hybridizes to the specific target polynucleotide and substantially does not hybridize to polynucleotides consisting of sequences which are not substantially complementary to the target polynucleotide. However, it will be recognized by those of skill that the minimum length of a polynucleotide desired for specific hybridization to a target polynucleotide will depend on several factors: G/C content, positioning of mismatched bases (if any), degree of uniqueness of the sequence as compared to the population of target polynucleotides, and chemical nature of the polynucleotide (e.g., methylphosphonate backbone, phosphorothiolate, etc.), among others.

Methods for conducting polynucleotide hybridization assays have been well developed in the art. Hybridization assay procedures and conditions will vary depending on the application and are selected in accordance with the general binding methods known in the art.

It is appreciated that the ability of two single stranded polynucleotides to hybridize will depend upon factors such as their degree of complementarity as well as the stringency of the hybridization reaction conditions.

A “ligand” is a molecule or a portion of a molecule that is recognized by a particular receptor. Examples of ligands that can be investigated by this invention include, but are not restricted to, agonists and antagonists for cell membrane receptors, toxins and venoms, viral epitopes, hormones, hormone receptors, peptides, enzymes, enzyme substrates, cofactors, drugs (e.g. opiates, steroids, etc.), lectins, sugars, polynucleotides, nucleic acids, oligosaccharides, proteins, and monoclonal antibodies.

A “receptor” is molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term “receptors” is used herein, no difference in meaning is intended. A “Ligand Receptor Pair” is formed when two macromolecules have combined through molecular recognition to form a complex. Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmitters is useful in the development of drugs which can be used in the treatment of disorders of neurotransmission.

c) Antibodies: For instance, the invention may be useful in investigating the ligand-binding site on the antibody molecule which combines with the epitope of an antigen of interest; determining a sequence that mimics an antigenic epitope may lead to the-development of vaccines of which the immunogen is based on one or more of such sequences or lead to the development of related diagnostic agents or compounds useful in therapeutic treatments such as for auto-immune diseases (e.g., by blocking the binding of the “anti-self” antibodies).

d) Nucleic Acids: Sequences of nucleic acids may be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant.

f) Hormone receptors: Examples of hormones receptors include, e.g., the receptors for insulin and growth hormone. Determination of the ligands which bind with high affinity to a receptor is useful in the development of, for example, an oral replacement of the daily injections which diabetics take to relieve the symptoms of diabetes. Other examples are the vasoconstrictive hormone receptors; determination of those ligands which bind to a receptor may lead to the development of drugs to control blood pressure.

g) Opiate receptors: Determination of ligands which bind to the opiate receptors in the brain is useful in the development of less-addictive replacements for morphine and related drugs.

A “fluorophore” or “fluorescent compound” can include, but is not limited to, a dye, intrinsically fluorescent protein, lanthanide phosphor, and the like. Dyes, for example, include rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃-coumarin-3-acetate, 7-NH₂-4CH₃-coumarin-3-acetate (AMCA), monobromobimane, pyrene trisulfonates, such as Cascade Blue, and monobromotrimethyl-ammoniobimane.

The term “complementary” refers to the topological compatibility or matching together of interacting surfaces of a ligand molecule and its receptor. Thus, the receptor and its ligand can be described as complementary, and furthermore, the contact surface characteristics are complementary to each other.

The term “wafer” means a semiconductor substrate. A wafer could be fashioned into various sizes and shapes. It could be used as a substrate for a microchip. The substrate could be overlaid or embedded with circuitry, for example, a pad, via, an interconnect or a scribe line. The circuitry of the wafer could also serve several purpose, for example, as microprocessors, memory storage, and/or communication capabilities. The circuitry can be controlled by the microprocessor on the wafer itself or controlled by a device external to the wafer.

Embodiments of the invention relate to a device and method that employ a semi-conductor device having an electrical sensor comprising as a transistor to detect the presence of a biomolecule in close proximity of the electrical sensor. The semiconductor device responds non-linearly to the presence of a bio-chemical species. The semiconductor device of the embodiments of the invention could be fabricated using three-dimensional integrated circuit process technology developed and can be made quite sensitive to the presence of different bio-chemicals or bio-molecules. The device includes transistors whose properties such as sub-threshold slope are changed during exposure of bio-molecules by bringing them in close proximity to the semiconductor device.

In the embodiments, the change in a property of the transistor when the biomolecule is brought in close proximity of the electrical sensor is a reflection of specific chemical and/or biological interactions and is detected by the electrical sensors, which can be part of an integrated on-chip device for performing chemical analysis and medical diagnostics. In specific embodiments, the electrical sensor could be a field effect transistor.

The embodiments of the invention further relate to a device and method where an array of electrical sensors is contained in a substrate. Signals from the electrical sensor are detected and collected by circuitries on the substrate or in a separate device. One application of the electrical sensor array of the invention is their use in a protein or DNA array for simultaneous multiple protein or DNA analysis. The substrate of the embodiments of the invention may be part of an integrated device that also serves as a microarray or macroarray, an integrated circuit, a microfluidic device, a MEMS, or a combination. Therefore, samples contained or processed by the device may be also analyzed by the integrated device and the signals processed for analysis.

A biological sample often contains many thousands or even more types of biomolecules and clinical diagnosis needs to measure multiple analytes for disease confirmation. Currently, each analyte is measured separately, which requires multiple samples from a patient. The sensors of the embodiment of this invention could be used as multiplex assays, in which multiple analytes can be measured at the same time.

In the embodiments of the invention, analytes that can be detected include antigens of all types, such as proteins, polysaccharides, and small molecules coupled to a protein. The specific bindings between antigens and their corresponding antibodies form the basis of immunoassays. Antibodies suitable for the embodiments of the invention include monoclonal antibodies, polyclonal antibodies, recombinant antibodies, random peptides and aptamers. Immunoassays suitable for the embodiments of the invention include solid-phase immunoassays based the sandwich principle and the competing principle. Also included are specific types of immunoassays such as enzyme-linked immunosorbent assay (ELISA) and electrochemiluminescence (ECL).

Analytes in the embodiments of the invention also include nucleic acids (DNA and RNA), which can form double-stranded molecules by hybridization, that is, complementary base pairing. The specificity of nucleic acid hybridization is such that the detection of molecular and/or nanomaterials binding events can be done through electrical readout of polarization changes caused by the interaction of charged target molecules (DNA, RNA, proteins, for example.) and chemically modified nanomaterials (carbon nanotubes, nanowires, nanoparticles functionalized with DNA, for example) with complementary molecular probes (DNA, RNA, anti-body, for example) attached to the electrodes (such as Au, Pt, for example). This specificity of complementary base pairing also allows thousands of hybridization to be carried out simultaneously in the same experiment on a DNA chip (also called a DNA array).

Molecular probes or capture molecules are immobilized on the surface of individually addressable electrical sensor arrays through surface functionalization techniques. The arrays of the embodiments of the invention could be a DNA array (collections of DNA probes on a shared base) comprising a dense grid of spots (often called elements or pads) arranged on a miniature support. Each spot could represent a different gene.

The capture molecule or probe in a DNA chip is usually hybridized with a complex RNA or cDNA target generated by making DNA copies of a complex mixture of RNA molecules derived from a particular cell type (source). The composition of such a target reflects the level of individual RNA molecules in the source. The intensities of the signals resulting from the binding events from the DNA spots of the DNA chip after hybridization between the probe and the target represent the relative expression levels of the genes of the source.

The DNA chip could be used for differential gene expression between samples (e.g., healthy tissue versus diseased tissue) to search for various specific genes (e.g., connected with an infectious agent) or in gene polymorphism and expression analysis. Particularly, the DNA chip could be used to investigate expression of various genes connected with various diseases in order to find causes of these diseases and to enable accurate treatments.

Using embodiments of the invention, one could find a specific segment of a nucleic acid of a gene, i.e., find a site with a particular order of bases in the examined gene. This detection could be performed by using a diagnostic polynucleotide made up of short synthetically assembled single-chained complementary polynucleotide—a chain of bases organized in a mirror order to which the specific segment of the nucleic acid would attach (hybridize) via A-T or G—C bonds.

The practice of the embodiments of the invention may employ, unless otherwise indicated, conventional techniques of micro-electronics, nanotechnology, organic chemistry, polymer technology, molecular biology (including recombinant techniques), cell biology, biochemistry, and immunology, which are within the skill of the art. Such conventional techniques include polymer array synthesis, immunoassays, hybridization, ligation, detection of molecules, such as antibodies and hybridization using a label. Specific illustrations of suitable techniques can be had by reference to the example herein below. However, other equivalent conventional procedures can, of course, also be used.

One embodiment of the invention relates to a device for detection of analytes. The device comprises an electrical sensor that changes one of its properties when a biomolecule is brought in close proximity of the electrical sensor. According to an embodiment of the invention, any sensor whose property such as sub-threshold slope is changed by change in the electrical charge of the environment within close proximity of the sensor can be used as the electrical sensor.

In a specific embodiment, the electrical sensor comprises an electromagnetic sensor, a transistor, an electrical resistance sensor, an electrical power sensor, a magnetism sensor, an electrical voltage sensor, or an electrical current sensor. Specific detection systems can be used as part or all of the electrical sensor include an ohmmeter, a multimeter, a galvanometer, an ammeter, a leaf electroscope, a voltmeter, a watt-hour meter, a magnetic compass, a fluxgate compass, or a magnetometer.

In another embodiment, the electrical sensor is a field effect transistor (FET). As discussed herein, an FET is a transistor that relies on an electric field to control the conductivity of a “channel” in a semiconductor material. An FET usually has three terminals, which are known as the gate, drain and source. A voltage applied between the gate and source terminals modulates the current between the source and drain terminals. In other words, in an FET, electrical current flows along a semiconductor path called the channel. At one end of the channel, there is an electrode called the source. At the other end of the channel, there is an electrode called the drain. Although the physical diameter of the channel is fixed for a given FET, its effective electrical diameter can be varied by the application of a voltage to a control electrode called the gate.

In one embodiment, the biomolecule could be brought in close proximity to of the gate area of the transistor such that the electrical charge of the biomolecule could create a voltage between the gate and source of the FET, thus creating an electrical current between the source and the drain of the FET. As discussed herein, both the mode of operation of the FET and strength of the current can be measured so that both the existence of the biomolecule and strength of the electrical charge on the biomolecule could be detected.

Many types of field effect transistor can be used in the embodiments of the invention, including both junction FET (JFET) and metal-oxide-semiconductor FET (MOSFET), as well as both the N-type semiconductor (N-channel) or P-type semiconductor (P-channel). In a specific embodiment, the FET is an MOSFET), a JFET, a metal-semiconductor FET (MESFET), or a high-electron-mobility (HEMFET).

In another embodiment of the invention, various nano-materials can be used in the field effect transistor, especially for serving as the channel between the source and drain, for enhanced sensitivity and selectivity. In a specific embodiment, the FET comprises a nanowire, a nanocrystal, or a nanotube, such as a single-walled or multi-walled carbon nanotube. The FET may also comprise a nanopillar, a nanogap, a patterned nanostructure.

In the embodiments of the invention, the analyte encompasses any compound, molecule or aggregate of interest for detection or analysis. Non-limiting examples of the analyte include an antibody, protein, peptide, receptor, antigen, DNA, RNA, polynucleotide, nucleic acid, carbohydrate, lipid, bacterium, macromolecule, allergen, carbohydrate, polysaccharide, glycoprotein, growth factor, cytokine, lipid, hormone, metabolite, cofactor, inhibitor, drug, pharmaceutical, poison, explosive, pesticide, nutrient, toxin, chemical warfare agent, biowarfare agent, biohazardous agent, infectious agent, prion, radioisotope, vitamin, carcinogen, mutagen, narcotic, heterocyclic aromatic compound, amphetamine, barbiturate, hallucinogen, waste product, and contaminant.

In one embodiment of the invention, the analyte comprises a biomolecule. More specifically, the analyte comprises an antigen, antibody, protein, peptide, virus, DNA, RNA, polynucleotide, nucleic acid, carbohydrate, lipid, bacterium, or macromolecule.

In the embodiments of the invention, an electrical charge change by bringing the biomolecule in close proximity of the sensor includes an electrical perturbation, impedance, current, voltage, or a photo-induced charge separation caused by the electrical charge change. The perturbance may be sensible by an electrical sensor in the forms of current, potential, impedance, or field effect.

In this regard, the embodiments of the invention can enable real-time detection of electrical charge changes caused by molecular events, such as biomolecular interactions discussed herein. In certain embodiments of the invention, the detection of an electrical perturbation, impedance, current, voltage, or a photo-induced charge separation by an FET is distance dependent. Specifically, when distance between the biomolecule and the surface of the FET is in the nanometer (nm) range, the sensitivity of the detection could be dependent upon the distance. In certain circumstances, the biomolecule far away from the sensor may not be detected. Thus, in a specific embodiment of the invention, the distance between the biomolecule and sensor could be less than 10 microns, preferably less than 1 micron, more preferably less than 1000 nanometer (nm), most preferably less than 100 nm.

According to another embodiment of the invention, the device or the electrical sensor is part of another device, e.g., an integrated circuit. Thus, in one embodiment, the electrical sensor is associated with a substrate, which may comprise a polymer, silicon or glass. In another embodiment, the substrate comprises a microarray, a macroarray, a multi-well plate, a microfluidic device, an integrated circuit, a MEMS or a combination thereof. The substrate may further comprise a microprocessor capable of processing signals or data detected by the electrical sensor.

In the embodiment of the invention, specific materials useful as the substrate include, but not limited to, polystyrene, polydimethylsiloxane (PDMS), silicon, glass, chemically functionalized glass, polymer-coated glass, nitrocellulose coated glass, uncoated glass, quartz, natural hydrogel, synthetic hydrogel, plastics, metals, and ceramics. The substrate may comprise any platform or device currently used for carrying out immunoassays, DNA or protein microarray analysis. Thus, the substrate may comprise a microarray or a macroarray, a multi-well plate, a microfluidic device, an integrated circuit, MEMS, or a combination thereof. Furthermore, the substrate may not be flat, and may comprise beads, particles, or other shaped objects.

In another embodiment of the invention, the substrate comprises a microprocessor comprising software or a hardware to process signal or data from the device or the electrical sensor. For example, the phase/intensity information as electrical signals generated by the sensor may be read to the microprocessor to transform and generate data, such as the type and quantity of a specific analyte detected.

In another embodiment of the invention, the substrate comprises a platform or device on which a chemical or biological assay is being performed. Specifically, the substrate may comprise a device for performing an immunoassay, such as an ELISA assay, wherein a sandwich type binding of antibody/antigen/antibody has been formed. The substrate may also comprise a DNA microarray assay, wherein a sandwich type capture molecule/target DNA/probe molecule binding has been formed. Thus, the device and detection according to the embodiments of the invention may be part of a larger device or process in which sequential and multiplex procedures may be performed.

In the embodiments of the invention, the microfluidic channel or multiple microfluidic channels may be part of the substrate, which may be an integrated device, such as an integrated circuit, a microfluidic device, or a MEMS. The microfluidic channels or their integrated devices can be made by using techniques known to skilled artisans or methods disclosed herein. For example, the microfluidic channels may be made by soft lithography method with poly-dimethyl siloxane. With these techniques it is possible to generate patterns with critical dimensions as small as 30 nm. These techniques use transparent, elastomeric polydimethylsiloxane (PDMS) “stamps” with patterned relief on the surface to generate features. The stamps can be prepared by casting pre-polymers against masters patterned by conventional lithographic techniques, as well as against other masters of interest. Several different techniques are known collectively as soft lithography.

Techniques used also include micromachining of silicon for microelectricalmechanical systems (MEMS), and embossing of thermoplastic with patterned quartz. Unlike conventional lithography, these techniques are able to generate features on both curved and reflective substrates and rapidly pattern large areas. A variety of materials could be patterned using the above techniques, including metals and polymers. The methods complement and extend existing nanolithographic techniques and provide new routes to high-quality patterns and structures with feature sizes of about 30 nm.

Standard lithography on silicone wafer or silica glass could also be used to fabricate the devices of the embodiments of this invention. Chambers or channels can be made from the devices, fluidic flow can be controlled by pressure gradient, electrical field gradient, gravity, heat gradient etc. The labels or label-conjugated molecules can also be separated by planar device with a single a plurality of chambers, where the surfaces are modified with polymers (polyethylene glycol (PEG)-dramatized compounds) that can minimize non-specific binding.

Embodiments of the present invention also encompass a device for analyte detection that comprises an array of electrical sensors and the associated complexes. Specifically, the device comprises an array of electrical sensors and a complex associated with a surface of each of at least a portion of the electrical sensors. In the embodiment, the complex comprises a label capable of creating an electrical charge change upon being exposed to radiation and the associated electrical sensor is capable of detecting the electrical charge change.

Thus, according to the embodiment, the device comprises an array of electrical sensors, such as field effect transistors, in a pre-designed pattern. In a specific embodiment, at least a portion of the electrical sensors are individually addressable. In other words, the type, location and electrical connection of the individual sensors are determined and controlled as desired. The embodiment enables the simultaneous and multiplex detection and analysis of analytes.

The embodiments of the invention relate to a device comprising a first substrate comprising a transistor; a second substrate; an insulating layer in between and adjoining the first and second substrates; and an opening within the second substrate, the opening being aligned with the transistor; wherein the transistor is configured to detect an electrical charge change within the opening. Preferably, the transistor is a field effect transistor (FET). Preferably, the FET is a metal-oxide-semiconductor FET (MOSFET), a junction FET (JFET), a metal-semiconductor FET (MESFET), or a high-electron-mobility (HEMFET). Preferably, the FET comprises a nanowire, a nanocrystal, a nanotube, a nanopillar, a nanogap, or a patterned nanostructure. Preferably, the EFT comprises a single-walled carbon nanotube. Preferably, the first and second substrates independently comprise a polymer, silicon or glass. Preferably, the first or second substrate comprises a silicon wafer. Preferably, the first and second substrates independently comprise a microarray, a macroarray, a multi-well plate, a microfluidic device, an integrated circuit, a MEMS or a combination thereof.

The device could further comprise a microprocessor capable of processing signals or data produced by the transistor. Preferably, the first substrate is attached to a supporting substrate. the attachment is through a bonding layer. Preferably, the first substrate is substantially flat and has a thickness of from about 10 nm to about 1.0 mm. Preferably, the insulating layer comprises silicon oxide. Preferably, the insulating layer has a thickness of from about 5.0 nm to about 100 nm. Preferably, the second substrate is substantially flat and has a thickness of from about 0.5 μm to about 10 mm. Preferably, the opening is through the thickness direction of the second substrate. Preferably, the space occupied by the opening comprises a cuboid, a cylinder, a prism, or a frustum. Preferably, the dimension of the opening is from about 10 nm to about 5 μm. Preferably, the transistor is an FET and the opening is aligned with the channel region of the FET. Preferably, the electrical charge change comprises an electrical perturbation, impedance, current, voltage, or a photo-induced charge separation. Preferably, an inside surface of the opening is functionalized to facilitate molecular binding. Preferably, the electrical charge change is created by a molecular binding event on or near an inside surface of the opening. Preferably, the molecular binding event comprises binding of a first binding partner on the inside surface of the opening and binding of a second binding partner to the first binding partner. Preferably, the first or second binding partner comprises a biomolecule. Preferably, the first binding partner comprises an antibody, an antigen, a receptor, a ligand, a protein, a peptide, a virus, a bacterium, a carbohydrate, a lipid, a polynucleotide, a nucleic acid or a macromolecule. Preferably, the second binding partner comprises an antigen, an antibody, a protein, a peptide, a virus, a bacterium, a carbohydrate, a lipid, a polynucleotide, a nucleic acid or a macromolecule. Preferably, the second binding partner comprises an antigen and the first binding partner comprises an antibody to the antigen. Preferably, the second binding partner comprises a peptide and the first binding partner comprises a receptor or ligand to the peptide. Preferably, the second binding partner comprises a first polynucleotide and the first binding partner comprises a complementary polynucleotide of the first polynucleotide.

Another embodiment of the invention is directed to a method comprising providing a substrate comprising a first part, a second part, and an insulating layer in between and adjoining the first and second parts; fabricating a transistor on the first part; and fabricating an opening within the second part, the opening being aligned with the transistor; wherein the transistor is configured to detect an electrical charge change within the opening. Preferably, the substrate comprises a silicon wafer. Preferably, the providing of the substrate comprises implanting oxygen ions into a predetermined region of the substrate to create the insulating layer, the insulating layer separating the substrate into the first and second parts. Preferably, the providing of the substrate comprises providing a first substrate and a second substrate; oxidizing a surface of the first and second substrates; and combining the first and second substrates through the oxidized surfaces; wherein the first substrate forms the first part, the second substrate forms the second part, and the combined oxidized surfaces form the insulating layer.

In one variation, the above method could further comprise attaching a supporting substrate to the first substrate. Preferably, the attaching is through a bonding means. The above method could further comprise independently fabricating a microarray, a macroarray, a multi-well plate, a microfluidic device, an integrated circuit, a MEMS, or a combination thereof on the first and second parts. The above method could further comprise fabricating a microprocessor on the first or second part, the microprocessor being capable of processing signals or data produced by the transistor. The above method could further comprise thinning the first or the second part. Preferably, the first part is substantially flat and has a thickness of from about 10 nm to about 1.0 mm. Preferably, the insulating layer has a thickness of from about 5.0 nm to about 100 nm. Preferably, the second part is substantially flat and has a thickness of from about 1.0 μm to about 10 mm. Preferably, the opening is through the thickness direction of the second substrate. Preferably, the transistor is an FET and the opening is aligned with the channel region of the FET. In one embodiment, the above method could further comprise funtionalizing an inside surface of the opening to facilitate molecular binding.

Yet other embodiments of the invention relate to a method comprising providing a device comprising a first substrate comprising a transistor, a second substrate, an insulating layer in between and adjoining the first and second substrates; and an opening within the second substrate, the opening being aligned with the transistor; providing an analyte on or near an inside surface of the opening; and detecting an electrical charge change on or near the inside surface of the opening using the transistor. The method could further comprise processing signals or data produced by the transistor. Preferably, the electrical charge change comprises an electrical perturbation, impedance, current, voltage, or a photo-induced charge separation. Preferably, an inside surface of the opening is functionalized to facilitate molecular binding. Preferably, the electrical charge change is created by a molecular binding event on or near an inside surface of the opening. The method could further comprise immobilizing a binding partner on the inside surface of the opening. Preferably, the providing of the analyte comprises binding the analyte to the binding partner. Preferably, the binding partner or the analyte comprises a biomolecule. Preferably, the binding partner comprises an antibody, an antigen, a receptor, a ligand, a protein, a peptide, a virus, a bacterium, a carbohydrate, a lipid, a polynucleotide, a nucleic acid or a macromolecule. Preferably, the analyte comprises an antigen, an antibody, a protein, a peptide, a virus, a bacterium, a carbohydrate, a lipid, a polynucleotide, a nucleic acid or a macromolecule. Preferably, the analyte comprises an antigen and the binding partner comprises an antibody to the antigen. Preferably, the analyte comprises a peptide and the binding partner comprises a receptor or ligand to the peptide. Preferably, the analyte comprises a first polynucleotide and the binding partner comprises a complementary polynucleotide of the first polynucleotide.

Yet other embodiments of the invention relate to a device comprising a first substrate, a second substrate, and an insulating layer in between and adjoining the first and second substrates; wherein the first substrate comprises an array of transistors and the second substrate comprises an array of openings, each of at least a portion of the openings being aligned with one of the transistors. Preferably, each of at least a portion of the transistors is capable of detecting an electrical charge change within the opening aligned with the transistor. Preferably, at least a portion of the transistors are field effect transistors (FETs). Preferably, at least a portion of the transistors are individually addressable. Preferably, an inside surface of at least a portion of the openings are functionalized to facilitate molecular binding. Preferably, an inside surface of each of at least a portion of the openings is bonded with one or more binding partners. Preferably, the binding partners bonded with a single opening comprise the same molecules. Preferably, at least two of the binding partners bonded with a single opening comprise different molecules. Preferably, the binding partners bonded with at least two of the openings comprise the same molecules. Preferably, the binding partners bonded with at least two of the openings comprises different molecules.

Yet other embodiments of the invention relate to a device comprising a substrate having a front side and a back side, an array of sensor nodes on the first side of the substrate and via openings through the thickness of the substrate, wherein at least some of the sensor nodes comprise a probe molecule functionalized though the via opening from the back side of the substrate. The device could further comprise a peripheral logic on a start wafer for column and row selection to access a particular sensor node. The device could further comprise a read-out circuit comprising a CMOS logic having a sense amplifier to detect a current change when an analyte molecule is dispersed into the via openings.

The embodiments of the invention are now explained with the following examples.

### EXAMPLES

### Example 1

### A Sensor of the Embodiments of the Invention

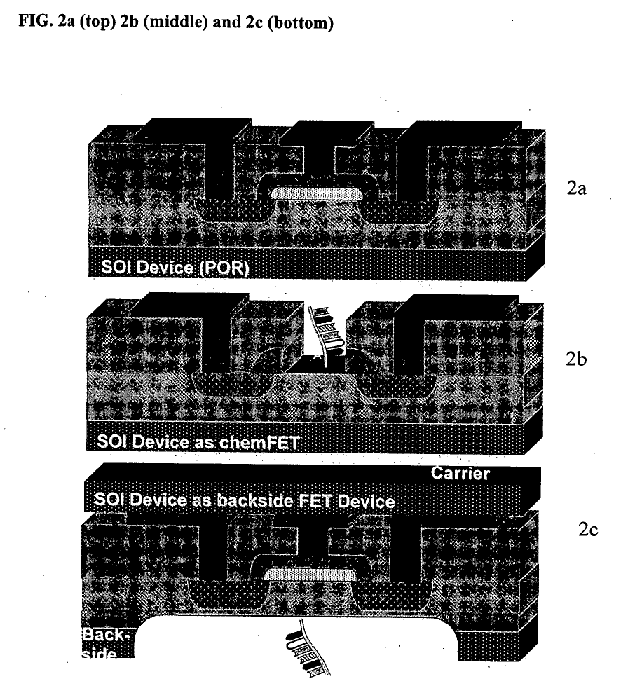

A sensor of the embodiments of the invention is shown in FIG. 2, which shows SOIFET (Silicon on Insulator Field Effect Transistor) as a bio-chemical sensor. FIG. 2a shows a plan of records a SOI FET used as logic transistors for microprocessor applications. A feature of a SOI FET is an extremely thin silicon fin sitting on top of a buried oxide; the Si film thickness is set by the gate length of the transistor-smaller the gate length, the thinner the Si film needs to be to maintain electrostatics in the channel. FIG. 2b shows a SOI device can act as a sensor by exposing the bio-chemical to the channel region of the transistor. The charge in the bio-chemical alters the transport properties of the transistor device and thus acts as a sensor. FIG. 2c illustrates a SOIFET sensor manufactured using 3-D wafer stacking technologies.

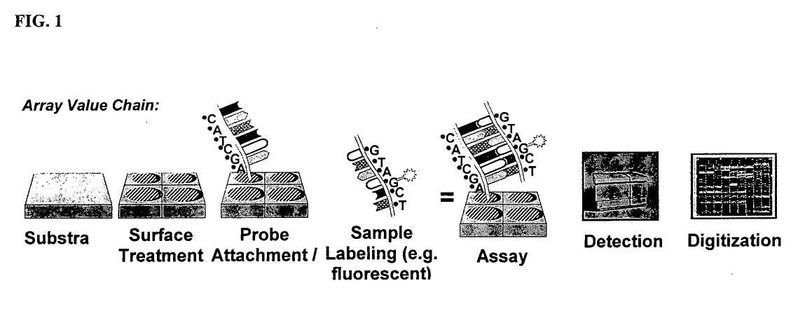

FIG. 1c shows the final device that is used as the SOIFET sensor. The MOSFET transistor processing is first completed on the SOI substrate. After that it is flipped and bonded to the handle wafer using Cu bonds for mechanical support. The SOI wafer substrate is then thinned down from the top to a thickness ranging between 0.5 micron to 5 micron. After a pattering step, via openings are created in the thinned substrate and etched down to the buried oxide, creating through silicon vias. In some cases, the buried oxide layer can be further thinned down using a timed wet etch process.

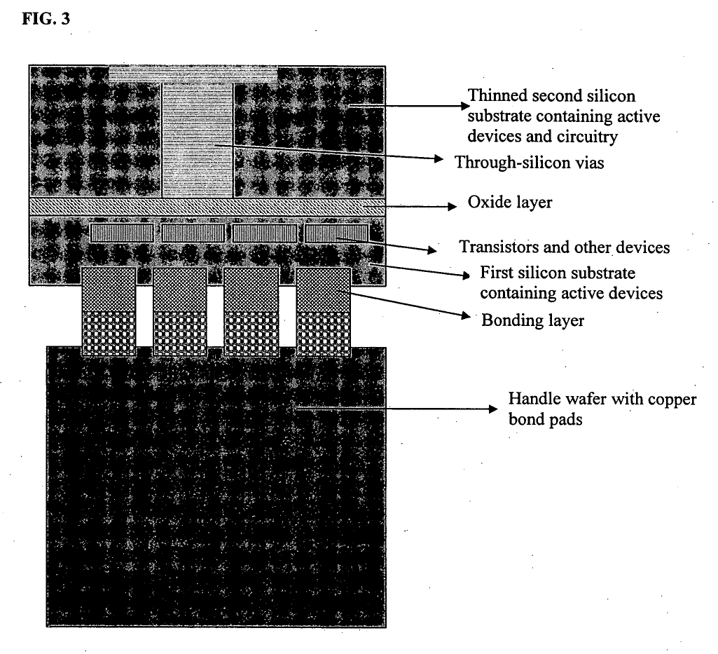

The wafer with the SOI device could be attached to a handle wafer using a copper bond layer as shown in FIG. 3. The device wafer could then be thinned to a few microns and subsequently through-silicon vias (openings) could be fabricated. The bio-chemicals could be dispersed into the vias and they would alter the device properties. Thereby, the semiconductor device of the embodiments of the invention could act as a sensor.

Referring to FIG. 3, the transistors and other devices are fabricated in a first silicon-containing substrate on an insulating layer (e.g., oxide layer) on a second silicon-containing substrate. A dummy bonding layer made of copper bumps is fabricated on top of the insulating layer or the first silicon-containing substrate. Copper bonds are created in the dielectric passivation layer for both the active SOI wafer and the handle wafer. The handle wafer with a similar bonding layer, which is the copper layer, is precisely aligned and bonded (e.g., by thermo-compression bonding) to the first silicon-containing substrate such that the another wafer serves the purpose of a handle wafer, which provides mechanical support during the grinding and etch-back process. After bonding, the second silicon-containing substrate, which is the SOI substrate wafer containing the active transistors, is thinned down to a few microns by mechanical grinding or etching. Through silicon vias corresponding to the transistor device are fabricated by etching. The silicon is etched selectively until the buried oxide layer is reached. At this point the buried oxide layer can be further thinned to a thickness ranging from 50 to 500 Angstroms. This will serve as the back gate oxide to the transistor channel. The biomolecule to be detected can be dispersed into this via and the charge associated with this biomolecule would alter the transport properties of the sensor.

### Example 2

### Effect of the Back Substrate Bias on the Channel Transport of the Sensor of the Embodiment of the Invention

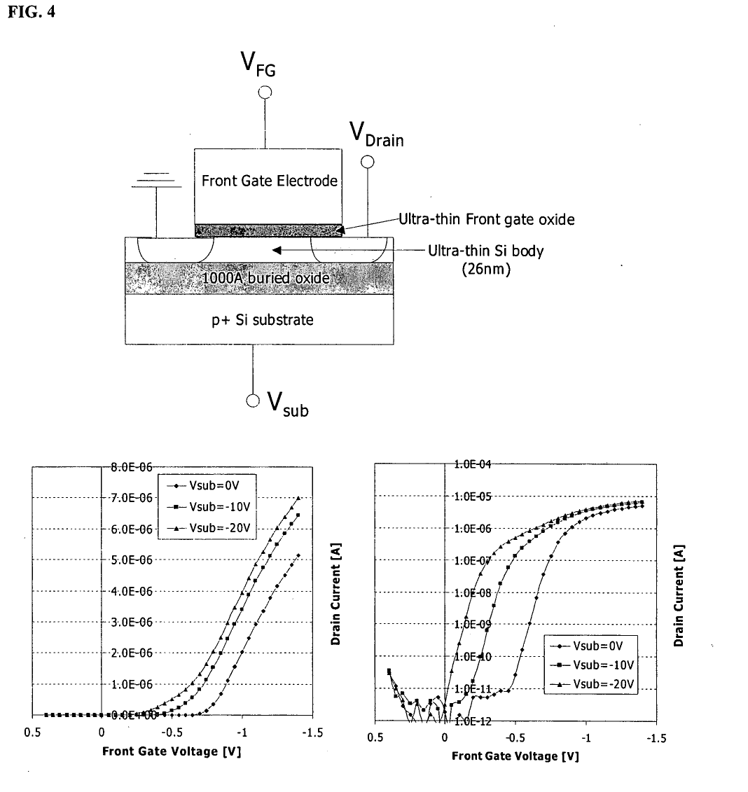

FIG. 4 (top) shows a schematic of a thin body SOI device which could be incorporated in the sensor of the embodiments of the invention. FIG. 4 (bottom) shows the effect of the back substrate bias voltage on the channel transport of a p type transistor-containing device. FIG. 4 shows that the substrate bias voltage could be coupled to channel and alter threshold voltage of the device, thereby affecting the drive current.

### Example 3

### A Microarray with a Sensor of the Embodiments of the Invention

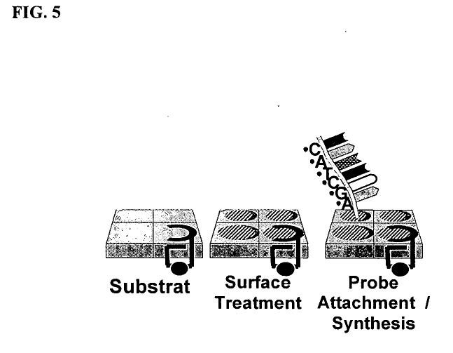

FIG. 5 shows various steps in the method of making a microarray with a sensor of the embodiments of the invention. The sensor of FIG. 3 will form one node in an array of nodes. At least some, preferably each, of these sensor nodes can be functionalized with a probe molecule of unique feature through the back side through silicon via openings. The density of the array will be determined by the back side via size and spacing and the alignment tolerance. There will be peripheral logic built out of CMOS logic technology in the SOI start wafer for column and row selection to access a particular sensor node; the read-out circuit is also realized in CMOS logic using a sense amplifier to detect the current (Iread) changes when an analyte molecule is dispersed into the via openings]

### Example 4

### A Method of Detecting and Analyzing a Biomolecule by the Embodiments of the Invention

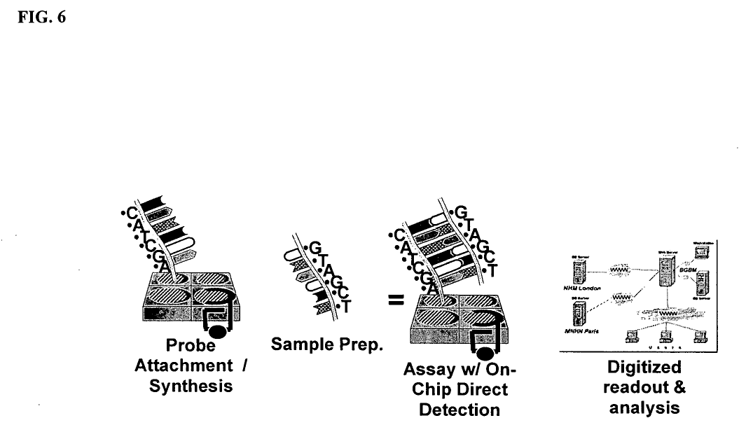

FIG. 6 shows a method of detecting and analyzing a biomolecule by a microarray having a sensor of the embodiments of the invention. The method of sample preparation, hybridization, on-chip direct detection and digitized readout and analysis are standard techniques well known to persons skilled in this art.

This application discloses several numerical range limitations that support any range within the disclosed numerical ranges even though a precise range limitation is not stated verbatim in the specification because the embodiments of the invention could be practiced throughout the disclosed numerical ranges. Further, the entire disclosure of the patents and publications referred in this application, if any, are hereby incorporated herein in entirety by reference.

1. A device comprising:
a first substrate comprising a transistor;
a second substrate;
an insulating layer in between and adjoining the first and second substrates; and
an opening through the second substrate, the opening being aligned with the transistor;
wherein the transistor is configured to detect an electrical charge change within the opening.

2. The device of claim 1, wherein the transistor is a field effect transistor (FET).

3. The device of claim 2, wherein the FET is a metal-oxide-semiconductor FET (MOSFET), a junction FET (JFET), a metal-semiconductor FET (MESFET), or a high-electron-mobility (HEMFET).

4. The device of claim 2, wherein the FET comprises a nanowire, a nanocrystal, a nanotube, a nanopillar, a nanogap, or a patterned nanostructure.

5. The device of claim 4, wherein the FET comprises a single-walled carbon nanotube.

6. The device of claim 1, wherein the first and second substrates independently comprise a polymer, silicon or glass.

7. The device of claim 1, wherein the first or second substrate comprises a silicon wafer.

8. The device of claim 1, wherein the first and second substrates independently comprise a microarray, a macroarray, a multi-well plate, a microfluidic device, an integrated circuit, a MEMS or a combination thereof.

9. The device of claim 1, further comprising a microprocessor capable of processing signals or data produced by the transistor.

10. The device of claim 1, wherein the first substrate is attached to a supporting substrate.

11. The device of claim 10, wherein the attachment is through a bonding layer.

12. The device of claim 1, wherein the first substrate is substantially flat and has a thickness of from about 10 nm to about 1.0 mm.

13. The device of claim 1, wherein the insulating layer comprises silicon oxide.

14. The device of claim 1, wherein the insulating layer has a thickness of from about 5.0 nm to about 100 nm.

15. The device of claim 1, wherein the second substrate is substantially flat and has a thickness of from about 0.5 μm to about 10 mm.

16. The device of claim 15, wherein the opening is through the thickness direction of the second substrate.

17. The device of claim 15, wherein the space occupied by the opening comprises a cuboid, a cylinder, a prism, or a frustum.

18. The device of claim 17, wherein the dimension of the opening is from about 10 nm to about 5 μm.

19. The device of claim 1, wherein the transistor is an FET and the opening is aligned with the channel region of the FET.

20. The device of claim 1, wherein the electrical charge change comprises an electrical perturbation, impedance, current, voltage, or a photo-induced charge separation.

21. The device of claim 1, wherein an inside surface of the opening is functionalized to facilitate molecular binding.

22. The device of claim 1, wherein the electrical charge change is created by a molecular binding event on or near an inside surface of the opening.

23. The device of claim 22, wherein the molecular binding event comprises binding of a first binding partner on the inside surface of the opening and binding of a second binding partner to the first binding partner.

24. The device of claim 23, wherein the first or second binding partner comprises a biomolecule.

25. The device of claim 23, wherein the first binding partner comprises an antibody, an antigen, a receptor, a ligand, a protein, a peptide, a virus, a bacterium, a carbohydrate, a lipid, a polynucleotide, a nucleic acid or a macromolecule.

26. The device of claim 23, wherein the second binding partner comprises an antigen, an antibody, a protein, a peptide, a virus, a bacterium, a carbohydrate, a lipid, a polynucleotide, a nucleic acid or a macromolecule.

27. The device of claim 23, wherein the second binding partner comprises an antigen and the first binding partner comprises an antibody to the antigen.

28. The device of claim 23, wherein the second binding partner comprises a peptide and the first binding partner comprises a receptor or ligand to the peptide.

29. The device of claim 23, wherein the second binding partner comprises a first polynucleotide and the first binding partner comprises a complementary polynucleotide of the first polynucleotide.

30. A method comprising:
providing a substrate comprising a first part, a second part, and an insulating layer in between and adjoining the first and second parts;
fabricating a transistor on the first part; and
fabricating an opening within the second part, the opening being aligned with the transistor;
wherein the transistor is configured to detect an electrical charge change within the opening.

31. The method of claim 30, wherein the substrate comprises a silicon wafer.

32. The method of claim 30, wherein the providing of the substrate comprises implanting oxygen ions into a predetermined region of the substrate to create the insulating layer, the insulating layer separating the substrate into the first and second parts.

33. The method of claim 30, wherein the providing of the substrate comprises:
providing a first substrate and a second substrate;
oxidizing a surface of the first and second substrates; and
combining the first and second substrates through the oxidized surfaces;
wherein the first substrate forms the first part, the second substrate forms the second part, and the combined oxidized surfaces form the insulating layer.

34. The method of claim 30, further comprising attaching a supporting substrate to the first substrate.

35. The method of claim 34, wherein the attaching is through a bonding means.

36. The method of claim 30, further comprising independently fabricating a microarray, a macroarray, a multi-well plate, a microfluidic device, an integrated circuit, a MEMS, or a combination thereof on the first and second parts.

37. The method of claim 30, further comprising fabricating a microprocessor on the first or second part, the microprocessor being capable of processing signals or data produced by the transistor.

38. The method of claim 30, further comprising thinning the first or the second part.

39. The method of claim 30, wherein the first part is substantially flat and has a thickness of from about 10 nm to about 1.0 mm.

40. The method of claim 30, wherein the insulating layer has a thickness of from about 5.0 nm to about 100 nm.

41. The method of claim 30, wherein the second part is substantially flat and has a thickness of from about 1.0 μm to about 10 mm.

42. The method of claim 39, wherein the opening is through the thickness direction of the second substrate.

43. The method of claim 30, wherein the transistor is an FET and the opening is aligned with the channel region of the FET.

44. The method of claim 30, further comprising funtionalizing an inside surface of the opening to facilitate molecular binding.

45. A method comprising:
providing a device comprising a first substrate comprising a transistor,
a second substrate, an insulating layer it between and adjoining the first and second substrates; and an opening within the second substrate, the opening being aligned with the transistor;
providing an analyte on or near an inside surface of the opening; and
detecting an electrical charge change on or near the inside surface of the opening using the transistor.

46. The method of claim 45, further comprising processing signals or data produced by the transistor.

47. The method of claim 45, wherein the electrical charge change comprises an electrical perturbation, impedance, current, voltage, or a photo-induced charge separation.

48. The method of claim 45, wherein an inside surface of the opening is functionalized to facilitate molecular binding.

49. The method of claim 45, wherein the electrical charge change is created by a molecular binding event on or near an inside surface of the opening.

50. The method of claim 49, further comprising immobilizing a binding partner on the inside surface of the opening.

51. The method of claim 50, wherein the providing of the analyte comprises binding the analyte to the binding partner.

52. The method of claim 51, wherein the binding partner or the analyte comprises a biomolecule.

53. The method of claim 50, wherein the binding partner comprises an antibody, an antigen, a receptor, a ligand, a protein, a peptide, a virus, a bacterium, a carbohydrate, a lipid, a polynucleotide, a nucleic acid or a macromolecule.

54. The method of claim 51, wherein the analyte comprises an antigen, an antibody, a protein, a peptide, a virus, a bacterium, a carbohydrate, a lipid, a polynucleotide, a nucleic acid or a macromolecule.

55. The method of claim 52, wherein the analyte comprises an antigen and the binding partner comprises an antibody to the antigen.

56. The method of claim 52, wherein the analyte comprises a peptide and the binding partner comprises a receptor or ligand to the peptide.

57. The method of claim 52, wherein the analyte comprises a first polynucleotide and the binding partner comprises a complementary polynucleotide of the first polynucleotide.

58. A device comprising:
a first substrate, a second substrate, and an insulating layer in between and adjoining the first and second substrates; wherein the first substrate comprises an array of transistors and the second substrate comprises an array of openings, each of at least a portion of the openings being aligned with one of the transistors.

59. The device of claim 58, wherein each of at least a portion of the transistors is capable of detecting an electrical charge change within the opening aligned with the transistor.

60. The device of claim 58, wherein at least a portion of the transistors are field effect transistors (FETs).

61. The device of claim 58, wherein at least a portion of the transistors are individually addressable.

62. The device of claim 58, wherein an inside surface of at least a portion of the openings are functionalized to facilitate molecular binding.

63. The device of claim 58, wherein an inside surface of each of at least a portion of the openings is bonded with one or more binding partners.

64. The device of claim 63, wherein the binding partners bonded with a single opening comprise the same molecules.

65. The device of claim 63, wherein at least two of the binding partners bonded with a single opening comprise different molecules.

66. The device of claim 63, wherein the binding partners bonded with at least two of the openings comprise the same molecules.

67. The device of claim 63, wherein the binding partners bonded with at least two of the openings comprises different molecules.

68. A device comprising a substrate having a front side and a back side, an array of sensor nodes on the first side of the substrate and via openings through the thickness of the substrate, wherein at least some of the sensor nodes comprise a probe molecule functionalized though the via opening from the back side of the substrate.

69. The device of claim 68, further comprising a peripheral logic on a start wafer for column and row selection to access a particular sensor node.

70. The device of claim 69, further comprising a read-out circuit comprising a CMOS logic having a sense amplifier to detect a current change when an analyte molecule is dispersed into the via openings.
